(19) United States
(12) Patent Application Publication
(10) Pub. No.: US 2019/0116816 A1
(43) Pub. Date: Apr. 25, 2019

(54) SYSTEM FOR REGISTRATION AND PRESENTATION OF PERFORMANCE DATA TO AN OPERATOR

(71) Applicant: Teknologisk Institut, Taastrup (DK)

(72) Inventors: Paul Andreas Holger Dirac, Taastrup (DK); Niels Toftelund Madsen, Taastrup (DK)

(21) Appl. No.: 16/092,067

(22) PCT Filed: Apr. 7, 2017
(86) PCT No.: PCT/EP2017/058357

§ 371 Date: Oct. 8, 2018

(30) Foreign Application Priority Data
Apr. 8, 2016 (DK) PA 2016 00215

(51) Int. Cl. A22C 17/00; A22B 5/00; G01N 33/12; G06T 19/00; G06Q 10/06

(52) U.S. Cl. CPC A22C 17/008; A22B 5/007; G06Q 10/06395; G06T 19/006; G01N 33/12

(57) ABSTRACT

The invention relates to a system and method for recording and presentation of performance data where the system comprises a transport system for transport of subjects or products to a measuring station which records one or more performance data for subjects which are transported by the transport system or wherein is entered one or more performance data related to the subjects which are transported by the transport system, an information system installed along with the transport system and where this information system releases an information related to one or more performance data for subjects with is transported by the transport system and where the released information is performed in immediate vicinity of or in contact with the respective subject for which the information is relevant.

The invention relates to a system for registration and presentation of performance data where performance data is recorded at a monitoring station and presented to an operator located downstream in relation to the monitoring station. The system can for instance be utilized at abattoirs for sorting and/or handling carcasses or parts thereof.

### BACKGROUND

At abattoirs and food firms sorting is performed of subjects such as meat product or carcasses which are transported at conveyors at high speed (e.g. 12,000 chickens/hour). Sorting is usually based on a control performed as human visual inspection where small subjects such as chicken with defects or other irregularities can be manually removed from the conveyor e.g. when discarded and larger subjects such as pig or cows/cattle can be directed to a side conveyor for subjects requiring further inspection or for being discarded, however, operators are not capable of examining all subjects properly at such high speed and some subjects with defects will proceed at the production chain. Operators performing visual inspection are also subjected to stress due to the high conveyor speed and the operators are not capable of performing the visual inspection for an entire working day and need breaks to rest the eyes and brain. To avoid or decrease the number of subjects with defects being sold e.g. to a company further processing the subjects, repeated inspection of the products at different location at the abattoir or food company may be necessary. Subjects with defects such as contamination with e.g. faeces or bile and which are not removed may cross contaminate subjects initially not being defective and may contaminate the production apparatus, and may eventually end at the consumer. Systems assisting visual inspection at high conveying speed are required.

WO 2008/102148 describes an apparatus and a method for handling food pieces, comprises subjecting a food piece to an X-ray analysis at an analysis position to detect a foreign object in the food piece. If a foreign object is detected, the food piece is transported from the analysis position to an operator and the operator is provided with an indication of the location of the foreign object in the food piece, where the step of providing an indication comprises providing a mark on the food piece such as an ink mark, a label or as projecting a visual representation of the X-ray image onto the food piece.

WO 2011/030042 describes a system and a method for aiding the manual sorting of objects. The system comprises: 1. A conveyor belt for conveying unsorted objects, 2. An analyzing station where a camera record an image of objects positioned in a matrix, 3. Equipment for comparing information of the image with a predetermined criterion, 4. Equipment for determining compliance or non-compliance for each subject of the matrix and deliver an identifier for each subject. 5. A sorting station where light is directed towards subjects identified as non-compliant with the predetermined criterion. At the sorting station objects are positioned in a matrix similar to when analyzed at the analyzing station and the conveying is stopped such that subjects indicated by light can manually be picked out. Such a system is not suitable for inspection during high conveying speed.

The sorting and handling of the products at abattoirs and food producing companies are dependent of preceding measurement and may consist of e.g. discarding of the subject or cutting away part of the subject. However, it is a challenge to present the performance data to a later part at the production chain, such as where an operator is positioned to handle the defective subjects.

The invention described herein is especially suitable to assist veterinarian or technical operators in the inspection or control procedure at abattoirs or food producing companies, such as in connection with the first veterinarian inspection of carcasses.

### SUMMARY

The invention relates to a system for recording and presentation of performance data, where the system comprises
A transport system for transport of subjects or products,
A measuring station which record one or more performance data for subjects or products which is transported by the transport system or into which one or more performance data related to the subject or products which is transported by the transport system is entered,
An information system installed along the transport system and where this information system emits or presents information related to one or more performance data for subjects or products which are transported by the transport system and where the emitting or presentation of information is performed in the immediate vicinity of or in contact with the respective subject or product for which the information is relevant and such that the emitted or presented information moves along the transport system together with the related subject or product being conveyed by the transport system.

The system is especially suitable to assist veterinarians or technical operators in the inspection procedure at abattoirs where e.g. as much as 1,200 pig carcasses or 12,000 chicken carcasses should be inspected each hour. The system may be capable of identifying defects and other irregularities at subjects/products and classify these subjects/products with defects into a category which may be indicated by the information system and may be visible for the veterinarians or technical operators.

Systems may be constructed for transporting and inspection of different kinds of subjects or products such as for carcasses or parts there off, other foodstuff such as fruit or vegetables, bottles, cans or any other subjects/products which should be inspected and where some action should be performed if a subject/product does not satisfy a predetermined quality.

The measuring station may comprise a vision based camera system with at least one camera. Images obtained by the at least one camera may be processed and analyzed by a processor such that defects or the presence of other visible quality lowering features on subjects/products can be analyzed and the system may classify the subjects/products with defects and/or features into a number of pre-determined categories.

The information system located along the transport system may be light bands or lamps located along with or above the transport system and the information which can be emitted or presented by the information system may be light and the color of the light may be correlated with the information which should be carried on by the system. Preferably the light moves along the information system with similar speed as the related subject or product moves in the transport system.

The invention further relates to a method for recording and presentation of performance data, the method may comprises
Recording performance data of a subject or product which is transported pass a measuring station or entering performance data into the measuring station,
Classifying the subjects or products based on the performance data into a number of predetermined categories for instance in three categories which can indicate ‘the subject/product is as it should be’, ‘the subject/product must be evaluated’ and ‘the subject/product shall be discarded’,
Emitting or presenting categorized performance data for a subject or product such that the categorized performance data follows the relevant subjects/products during a further transport and where the emitting or presentation of information is performed in the immediate vicinity of or in contact with the respective subject or product for which the information is relevant.

The emitted or presented information preferably moves along the transport system together with the related subject or product being conveyed by the transport system.

To minimize or prevent accumulation of subjects/products in the transport system the emitted or presented information preferably moves along the transport system together with the related subject or product being conveyed by the transport system and with the same speed as these subjects/products, such that the information relating to a subject/product without delay can be detected by an operator who subsequently can perform the required treatment of a subject/product and which treatment preferably is based on the received information.

Performance data may be obtained by a vision system and at least one image obtained by a camera of the vision system may be analyzed by an image processing system such that structures of the at least one image are weighted and/or identified and the subject/product are classified in relation to a predetermined number of categories. The classifying of subjects/products based on the performance data may be into e.g. three categories which each may be correlated with light of a specific color or with two colors together with no light, such that the three categories may be indicated by the information system close to a relevant subject/product.

The system and method described herein may be used for recording and presentation of performance data such as at an abattoir or at another food producing company.

### DETAILED DESCRIPTION

An aspect of the invention relates to a system for recording and presentation of performance data, where the system comprises
A transport system for transport of subjects or products,
At least one measuring station which record one or more performance data for subjects or products which are transported by the transport system or into which measuring station one or more performance data related to the subject or products which is transported by the transport system is entered,
An information system installed along the transport system and where this information system emits or presents information related to one or more performance data for subjects or products which are transported by the transport system and where the emitting or presentation of information is performed in the immediate vicinity of or in contact with the respective subject or product for which the information is relevant and such that the emitted or presented information moves along the transport system together with the related subject or product being conveyed by the transport system.

A transport system may be a conventional conveyor system such as for conveying suspended animal carcasses e.g. of pigs, cattle/cows, poultry, sheep, goats, deer, or it may be a conveyor system for conveying resting subjects/products e.g. parts of animal carcasses or other subjects/products such as those mentioned herein. A transport system may also be a conventional conveyor belt such as for conveying parts of animal carcasses or other subjects/products such as those mentioned herein. The transport system may also be a conveyor system for carrying suspended containers capable of holding subjects/products.

In an embodiment the transport system is for conveying carcasses or parts there off, bottles, cans, or other types of foodstuff such as fruit or vegetables.

A measuring station may be any station or system capable of obtaining information related to a subject or product being conveyed and/or a system into which information relating to a subject or product may be introduced electronically and/or manually. The system for recording and presentation of performance data may comprise at least one measuring station, such as two measuring systems. In a system with more than one measuring systems, these measuring systems are preferably positioned at different locations along the conveying path and/or obtain performance data relating to different sections of the subjects/products and/or uses different techniques or different types of measuring instruments to obtain performance data.

In an embodiment the at least one measuring station may comprise a vision based camera system with at least one camera.

The at least one measuring station may further comprise or be communication-wise connected to an input panel for entering data such as one or more performance date, which data or performance data may be obtained preferably before the relevant subject/product passes the measuring station and/or before the relevant subject/product passes the last control point in the production facility. At abattoirs such data or performance data obtained before the relevant subject/project passes the at least one measuring station may be any data obtained in relation to the carcass or the living animal before slaughter or during stunning and/or killing of the animal e.g. data relating to possible transport problems during live transportation from the animal farmer to the abattoir, any possible bruises recognized before stunning, or other defects or irregularities observed before the subject/product passes the measuring station.

The at least one measuring station may comprise or be communication-wise connected to a processor capable of analyzing obtained information, such as capable of analyzing digital images. At least one image may be obtained for each subject or product being conveyed, such at least one image may be analyzed e.g. by comparing to at least one reference image obtained from subjects or products which is of known category according to thresholds resulting in a predetermined number of categories e.g. three categories which may be: a) accepted level, b) non-accepted level and c) a level in-between which e.g. should be further controlled such as by an operator. The images may be analyzed for any relevant subject/product quality such as defects of the subject/product e.g. broken bones; discoloration e.g. bruises; foreign bodies e.g. plastic, metal; contaminations e.g. faeces; diseases e.g. abscesses. For each quality parameter the thresholds used by the processor may be adjusted due to a customer's accepted level of the specific quality parameter and/or due to veterinarian rules.

Measurements performed on carcasses or meat products can be control or quality measurements based on any sensor type and/or sensors of different types. As an example the at least one measure system can be based on vision. A system as described herein would especially be relevant where measurement data can be obtained for detection of conditions which is usually mentioned as ‘remarks’ and which is used by the veterinarian or meat inspection at abattoirs. Subjects/products categorized into the group ‘remarks’ should preferably be further examined and potentially handled to remove the detected conditions before the subject/product proceeds at the conveyor path at the abattoir. It is a challenge to present the performance data to a later part of the production chain. For instance must performance data in the veterinarian inspection be presented for a person such as a veterinarian who must take action dependent of the performance data.

Performance data which is recorded at at least one measure station can for example be simplified to one of three situations: 1) The product is okay, 2) the product needs further inspection or 3) the product is useless. These situations can be predefined based on evaluation in relation to a scale running from no presence of what is measured to increasing presence of what is measured and where the scale reaches a level of presence where discarding of the entire product must be performed. The scale or thresholds of the scale can be adjusted to the visual signs of defects and diseases which are known in the meat production such as at abattoirs and at meat firms.

As described the information which is emitted or presented by the information system may be based on subdivision of the subjects/products based on the performance data into categories as for instance subdivided into three categories which e.g. can indicate ‘product is as it should be’, ‘the product must be evaluated’ and ‘the product shall be discarded’ and where the information which is emitted can be light of the colors ‘green’, ‘yellow’ and ‘red’ respectively for the three categories. Other types of techniques forwarding the categorized information may also be possible such as different symbols, different letters or different numbers, or a combination of e.g. light and symbols/letters/numbers making it possible to categorize into more than two or three categories. Instead of light liquid-crystal display (LCD) monitors, displays, panels etc may be used or other techniques capable of communicating the category of a specific subject/product.

In parallel with a conveyor or a conveyor belt which transports carcasses or meat products or other products an information band may be arranged which can be based on diodes, laser light or equivalent. The information band can be located above and/or along the conveyor or conveyor belt. The invention may comprise an information system where information on an information band moves in parallel with the subject on the conveyor or conveyor belt. The information can for instance be a color which indicates what action is required by an operator further down the line. The information can be indicated in a simple way such as green=okay, red=useless and yellow=re-examination. The colors may be indicated as light in the information band. The colors may ‘run along’ the information band at the same speed as the subject/product is conveyed and the ‘running’ effect may occur when lighting items of the relevant color are turned on and off in the information band according to the status of the subject/product being in proximity to the specific location at the information band and such that the color related to a subject/product at all time is located exactly in connection with the relevant subject/product which preferably is under transport.

The information system may be of light bands or lamps located along with or above the transport system. For conveyor belts an information system based on light may be located at one or both sides of the conveyor belt and/or above the conveyor belt. The location of the information system may vary along a conveyor belt such that the light bands or lamps are located at a position making it comfortable and easy for an operator to see the light emitted from the light band or lamps and easily recognizes the information forwarded by the color of the light. For conveyors for suspended subjects/products an information system based on light may be located at one or both sides of the conveyor, above the conveyor or below the conveyor. Preferably the information system is located above the conveyor where the information system is expected to be lesser dirty than below the conveyor where drips from e.g. carcasses or meat products may be expected.

In a preferred embodiment the information is emitted or presented by the information system as light and a color of the light is correlated with the information which should be carried on by the system and the light moves along the information system with similar speed as the related subject or product moves in the transport system.

The information band can for instance be based on light band which is synchronized with the belt speed or laser light which moves together with the belt speed or spotlights which shine light on the subject/product with the color of relevance in relation to the present subject/product.

The information emitted by the information system may also be based on two, three or more types of signals e.g. colors and numbers; colors and letters; colors and symbols; colors, numbers and letters; colors, numbers and symbols; numbers, letters and symbols. Each type of signal e.g. color, number, letter, symbol etc may be used in a number of predetermined categories such as 3 colors, 3 numbers, 4 letters, 2 symbols and each signal type may forward specific information related to the subject/product of relevance. The number of signal types may be any relevant and the number of categories of each signal type may be predetermined by the food producing company incl. abattoirs. Each signal type may include its own information e.g. color can be 1) okay, 2) inspection required and 3) to be discarded e.g. by green, yellow and red, respectively. A number may relate to ‘inspection required’ and may describe different types of defects. A letter may relate to the type of operator which should inspect and/or handle the subject/product. A symbol may relate to the type of waste into which the subject/product should be sorted. Different categories and number of categories may be used for each used signal type.

To prevent faults at stop or drop of subjects an identification tool can be added which secure the pace is correct. This can for instance be tracing and monitoring of each tenth suspension hook.

The system can be extended with detailed information which can be viewed at a display or in a pair of spectacles or a screen at the individual operator.

The system may further comprise a unit for supplying an operator with further information, such a unit may be a pair of spectacles based on augmented reality which can be used by at least one operator who receives information from the at least one measuring station and where the operator simultaneous can receive information passed by the information system (e.g. by a green, yellow or red color) and in the spectacles the operator may view images of a subject that passes the operator together with information which superimpose the image of the subject such that the operator can receive more information e.g. coverage and/or location of what is recorded at the measuring station.

Augmented reality (AR) can be used for presentation of information. The invention thus comprises an equipment for transfer of information from at least one measure station to operators in the form of data suitable to be used in augmented reality and where the information is correlated with the subject/product e.g. carcass or meat product which passes the operator in question.

Another aspect of the invention relates to a method for recording and presentation of performance data, where the method may comprises
Recording performance data form a subject or product which is transported pass at least one measuring station or entering performance data into the at least one measuring station,
Classifying the performance data into a number of categories for instance in three categories which can indicate ‘the subject/product is as it should be’, ‘the subject/product must be evaluated’ and ‘the subject/product shall be discarded’,
Emitting or presenting categorized performance data for a subject or product such that the categorized performance data follows the relevant subjects/products during a further transport and where the emitting or presentation of information is performed in the immediate vicinity of or in contact with the respective subject or product for which the information is relevant and such that the emitted or presented information moves along the transport system together with the related subject or product being conveyed by the transport system.

The formulation that emitting or presentation of information can be in contact with the respective subject or product preferably does not mean physical contact at least for food or feed products which should not be contaminated. ‘In contact with’ means that the emitted or presented information may be positioned towards the subject/product e.g. by a light beam highlighting subjects/products selected through an analysis by a processor.

The method may comprise that the emitted or presented information moves along the transport system together with the related subject or product being conveyed by the transport system and with the same speed as these subjects/products, such that the information without delay can be detected by an operator who subsequently can perform the required treatment of a subject/product based on the received information.

In the method performance data may be obtained by at least one vision system where at least one image obtained by a camera of the at least one vision system can be analyzed by an image processing system such that structures of the at least one image are weighted in relation images of subjects/products of known categories. The weighting of images may be in relation to a scale including the end points ‘the subject/product is as it should be’ and ‘the subject/product shall be discarded’ and wherein the weighting results in categorizing the subject/product into e.g. at least three categories, where these categories may comprise ‘the subject/product is as it should be’, ‘the subject/product must be evaluated’ and ‘the subject/product shall be discarded’.

In the method the classifying of the subjects/products based on the performance data into e.g. three categories may be correlated with light of two colors together with no light, such that the three categories may be ‘no light’, ‘a bright light’ such as white or yellow light, and ‘a darker light’ such as blue or red light, or the classifying of the performance data into three categories may be correlated with light of three colors such as ‘green’, ‘yellow’ and ‘red’. The method may be further understood from the description herein in respect of the system.

The method may comprise that performance data and/or other information can be presented to an operator based on augmented reality such as in a pair of spectacles which is carried by the operator such that the operator receives further information than that received by the categorized performance data such as an image or constructed image of the respective subject/product and which image was recorded at the measuring station or which constructed image is based on image analysis of one or more images obtained at at least one measuring station.

The method may be performed with the system as described herein and may be used for control and categorizing of subjects/products such as carcasses or parts thereof, bottles, cans or other foodstuff e.g. fruit or vegetables. Such a control may be performed at any company e.g. at abattoirs or other food producing companies.

The described system and the described method may be used for all types of carcasses such as chicken, pigs, cattle/cows, sheep, goats, rabbits etc.

The described system and the described method can be used for all types of visual inspection matter at carcasses and meat products. However, other types of information can also be communicated in the described system, including information relating to the animal's youth or conditions earlier in the slaughtering process or in the process leading to production of meat products.

The described system and the described method are in particular suitable for use as a vision system for instance at abattoirs as a help to make diagnoses. The system can perform the first classification into categories of subjects/products such as described above. An operator and/or veterinarian may subsequent manually evaluate and handle the carcasses and/or meat product which are classified as ‘yellow’ (inspection required) and/or ‘red’ (to be discarded).

The described system and the described method may contribute to an improved classification of e.g. carcasses and/or meat products at abattoirs and in food producing companies.

In abattoirs the method described herein may comprise:
Entire carcasses of e.g. poultry, pigs or cows/cattle are prepared to be ready to become suspended in conveyors such as shackles, and conveying of the carcasses is initiated,
The carcasses are cut open such that the viscera are visible or are removed fully or partly,
Carcasses and the viscera passes at least one measuring system where at least one image of each of the carcasses and related viscera are obtained, however, a carcass and related viscera need not pass the same measuring system,
At least one image from each carcasses and related viscera is analyzed by image analysis by a processor which categorizes the carcasses into a number of categories,
An information band located in proximity to e.g. above the conveyor and which information band comprises light indicates the determined category for each carcass being conveyed such that the light ‘runs’ with similar speed as the carcasses are conveyed,
An operator such as a veterinarian recognizes the light color indicating ‘further inspection and/or handling is required’ and handle and/or inspect the relevant carcasses according to the requirements, preferably a veterinarian only inspects the carcasses and may enter further information into the system withholding or amending the category into which the carcass is categorized,
Another operator preferably downstream the line recognizes the light color indicating ‘the carcass should be discarded’ and removes the corresponding carcasses from the conveyor by lifting the carcass of the conveyor or removes the corresponding carcasses by direction the carcasses away from the conveyor path,
A further operator downstream the line recognizes the light color indicating ‘further inspection and/or handling is required’ and handles the carcass such as by cutting away bruises, and may afterwards enter information into the system what kind of handling is performed and/or change the category for the respective carcass to ‘the subject/product is as it should be’ e.g. corresponding to a green light,
The carcasses proceed further in the processes at the abattoir.

The described system and the described method may likewise be used in other areas of business for sorting such as sorting of bottles, cans and for other foodstuffs e.g. fruit and vegetable.

A further aspect of the invention relates to use of the system as described herein for recording and presentation of performance data such as at an abattoir or at another food producing company.

Yet another aspect of the invention relates to use of the method as described herein for recording and presentation of performance data such as at an abattoir or at another food producing company.

The system and method as described herein may also be used for identifying and indicating foreign bodies such as at and/or in meat products e.g. meat pieces, at and/or in minced meat, at and/or in shellfish etc. Such products may be analyzed by the system and method at the production line at any time such as straight after the meat products or meat pieces are produced and/or when the meat products or meat pieces are ready to be packaged e.g. when a meat product such as minced meat is arranged in a tray.

For shellfish the system and method as described herein may e.g. be used for sorting different species of shellfish and/or for identifying foreign bodies among/at/in the shellfish and where these two purposes may be performed by using a single measuring system for obtaining performance data or by using at least two measuring systems for obtaining performance data e.g. one measuring system for obtaining performance data related to species and one measuring system for obtaining performance data related to foreign bodies. If using a single measuring system the categories of the system may be: 1) Species A, 2) Species B or Other species, 3) Presence of foreign bodies, and these categories may be indicated at the information system by the colors green, yellow and red, respectively.

1. A system for recording and presentation of performance data, comprising:
transport system for transport of subjects or products,
at least one measuring station configured to record or receive one or more performance data for subjects or products transported by the transport system;
an information system installed along the transport system and configured to emit or present information related to the one or more performance data for the subjects or products transported by the transport system; wherein the emitting or presentation of information is performed in an immediate vicinity of or in contact with a respective subject or product of the subjects or products for which the information is relevant; and wherein the emitted or presented information moves along the transport system together with the respective subject or product being transported by the transport system.

2. The system according to claim 1, wherein the transport system comprises one or more of:
a conveyor system configured to convey suspended carcasses, parts of carcasses, or for carrying suspended configured to hold the subjects or products transported by the transport system;
a conveyor system configured to rest the subjects or products transported by the transport system, wherein the subjects or products transported by the transport system comprise one or more of: bottles, cans or foodstuff.

3. The system according to claim 1, wherein the at least one measuring station comprises a vision based camera system comprising at least one camera.

4. The system according to claim 1, wherein the information system comprises one or more of: a light band or a set of lamps located along or above the transport system.

5. The system according to claim 1, wherein the information which is emitted or presented by the information system is light, and wherein a color of the light is correlated with the information, and wherein the light moves along the information system with a similar speed as speed at which the related subject or product moves in the transport system.

6. The system according to claim 1, wherein the information which is emitted or presented by the information system is based on subdivision of the performance data into categories as for instance subdivided into three categories which e.g. can indicate ‘product is as it should be’, ‘the product must be evaluated’ and ‘the product shall be discarded’ and where the information which is emitted can be light of the colors ‘green’, ‘yellow’ and ‘red’ respectively for the three categories.

7. The system according to claim 1, further comprising an augmented reality unit configured to be used by at least one operator, wherein the the information system is configured to provide the information to the at least one operator, and wherein the augmented reality unit is configured to superimpose the information about the respective subject or product over an image of the subject or product.

8. A method for recording and presentation of performance data, said method comprises
recording performance data of a subject or product transported past a measuring station or entering performance data relating to the subject or product into the measuring station,
classifying the performance data into a number of categories;
emitting or presenting the categorized performance data for the subject or product such that the categorized performance data follows the relevant subject or product during a further transport; and wherein the emitting or presentation of the categorized performance data is performed in an immediate vicinity of or in contact with the respective subject or product for which the categorized performance data is relevant

9. The method according to claim 8, wherein the emitted or presented categorized performance data moves along the transport system together with the related subject or product being conveyed by the transport system and with a same speed at which the subject or product moves along the transport system, wherein the categorized performance data is detectable by an operator without delay.

10. The method according to claim 8, further comprising:
obtaining, by a vision system, the performance data; and
analyzing, by an image processing system, at least one image obtained by a camera of the vision system.

11. The method according to claim 8, wherein each category is correlated with a light of a specific color.

12. The method according to claim 8, further comprising:
presenting wherein the classified performance data or other information to an operator using augmented reality using an image recorded at the measuring station.

13. (canceled)

14. The system of claim 1, wherein the recording and presentation of the performance data occurs at an abattoir or at another food producing company.

15. The method according to claim 8, wherein the recording and emitting or presenting occur at an abattoir or at another food producing company.
